(19) United States
(12) Patent Application Publication
(10) Pub. No.: US 2013/0224729 A1
(43) Pub. Date: Aug. 29, 2013

(54) Biodetection Methods and Compositions

(71) Applicants: George M. Church, Brookline, MA (US); Adeyemi Adesokan, Cambridge, MA (US)

(72) Inventors: George M. Church, Brookline, MA (US); Adeyemi Adesokan, Cambridge, MA (US)

(73) Assignee: President and Fellows of Harvard College, Cambridge, MA (US)

(21) Appl. No.: 13/389,839

(22) PCT Filed: Aug. 12, 2010
(86) PCT No.: PCT/US10/45269

§ 371 Date: Mar. 29, 2012

Related U.S. Application Data
(60) Provisional application No. 61/233,306, filed on Aug. 12, 2009.

(51) Int. Cl. C12Q 1/68

(52) U.S. Cl. CPC C12Q 1/6895; C12Q 1/6888; C12Q 1/689
USPC 435/5; 435/6.11

(57) ABSTRACT

Diagnostic methods and compositions for detecting biological material are provided.

### RELATED APPLICATION

This application claims priority from U.S. provisional patent application No. 61/233,306, filed Aug. 12, 2009, which is hereby incorporated herein by reference in its entirety for all purposes.

### STATEMENT OF GOVERNMENT INTERESTS

This invention was made with government support under HG003170 awarded by the National Institutes of Health. The government has certain rights in the invention.

### BACKGROUND

1. Field of the Invention

Embodiments of the present invention relate in general to the use of molecular inversion probe technology coupled with database technology for detecting biological material.

2. Description of Related Art

Biodetection methods (e.g., pathogen detection methods) are critical in a variety of arenas such as bio-security, microbial forensics, agriculture, hospital diagnostics, clinical diagnostics, public health (e.g., the detection and identification of allergens, viruses, fungi and emerging diseases, as well as strain typing in food safety) and the like.

Current biodetection technologies include DNA-based detection methods (e.g., real time PCR, microarray hybridization), ligand-based methods (e.g., peptides, antibodies, single chain variable fragments, aptamers and carbohydrates), spectroscopy-based sensor methods (e.g., vibrational signatures of physicochemical properties of organisms), and transduction methods (e.g., cell environment sensing such as G protein signaling cascades in neutrophils and photon detection by rhodopsins). These current technologies suffer many drawbacks. For example, they are unable to detect previously unknown, mutant engineered or drug resistant organisms, they require large amounts of sample for detection, and they provide limited resolution in strain identification and are unable to discriminate between pathogens and non-virulent near neighbor species. Accordingly, novel methods of biodetection are needed.

### SUMMARY

Methods for determining a phenotype of an organism in a sample are provided. The methods include the steps of obtaining a sample, contacting the sample with a molecular inversion probe (MIP), wherein the MIP includes two regions of homology to a target nucleic acid sequence of interest in the organism and two probe amplification regions, wherein the two regions of homology are selected using a MIP database specific for the phenotype, hybridizing the MIP to the nucleic acid sequence of interest, converting the target nucleic acid sequence of interest to circular DNA, amplifying the circular DNA, releasing the MIP from the amplified DNA, sequencing the amplified DNA, and determining whether a DNA sequence corresponding to the phenotype is present. In certain aspects, the organism is one or more of a bacterium (e.g., one or more of Y. pestis, Brucella, Avian pathogenic E. coli, Quinolone resistant E. coli, Rickettsiae, Group B Streptococci, Burkholderia mallie, Bordetalla parapertusis, drug resistant P. falciparum, M. tuberculosis, V. cholera, B. anthracia, E. faecium, F. tularensis, B. pertussis and methicillin resistant S. aureus), a virus (e.g., one or more of HIV-1, avian influenza and dengue virus), a fungus and a protist. In certain aspects, the amplification step is performed by rolling circle amplification (RCA). In other aspects, the sequencing step is performed by multiplex sequencing. In yet other aspects, the MIP database is a single nucleotide polymorphism (SNP) database, an antibiotic resistance gene database, a virulence gene database or any combinations thereof. In still other aspects, the phenotype is antibiotic resistance and/or virulence.

Advantages of embodiments of the present invention include efficiencies of economy and time. Embodiments of the present further provide a superior DNA sequence-based biodetection technology, the ability to detect previously unknown, mutant and/or engineered organisms, the ability to differentiate between pathogen and non-virulent “near neighbor” species, the ability to detect the appearance of polymorphisms, and a very efficient, time-saving multiplex approach.

Further features and advantages of certain embodiments of the present invention will become more fully apparent in the following description of the embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present invention will be more fully understood from the following detailed description of illustrative embodiments taken in conjunction with the accompanying drawings in which:

FIG. 1 schematically depicts molecular inversion probe technology (For a review, see Li et al. (2009) Science 324:1210; Ball et al. (2009) Nat. Biotech. 27:361; Porreca et al. (2007) Nat. Meth. 4:931).

FIG. 2 schematically depicts multiplex capture. Over 30,000 targets can be probed in a single reaction (Li et al., supra).

FIG. 3 schematically depicts important bacterial pathogens that can be detected by the methods described herein.

FIGS. 4A-4C schematically depict viral pathogens that can be detected by the methods described herein. (A) DNA viruses. (B) Retroid viruses. (C) Other viruses.

FIG. 5 schematically depicts eukaryotic pathogens that can be detected by the methods described herein.

FIG. 6 depicts protease and reverse transcriptase mutations for drug resistance surveillance that existed in 2009 (new mutations are depicted in bold).

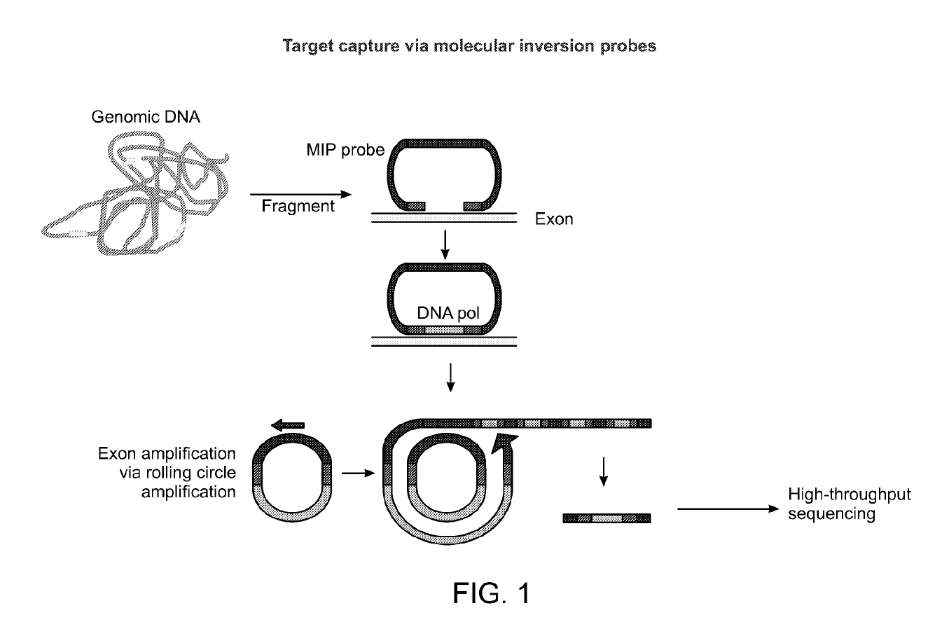

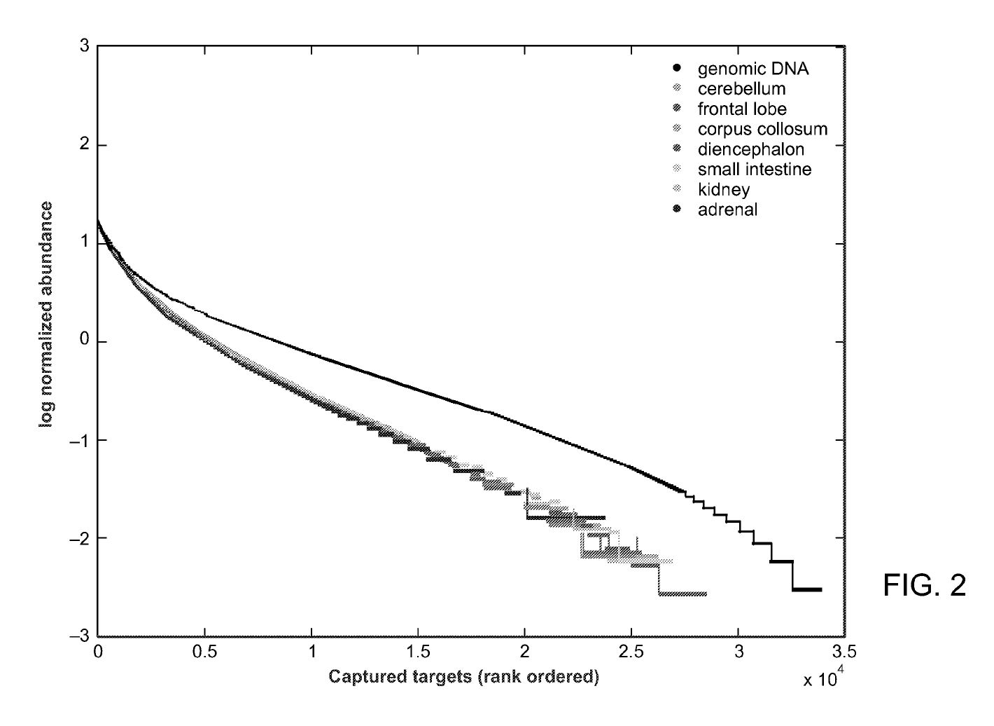

### DETAILED DESCRIPTION

Biodetection systems known in the art are not able to detect and identify new and/or engineered pathogenic strains. The biodetection methods described herein remedy these deficiencies in part by addressing important challenges for infectious disease identification sensitivity and breadth, and discriminating between pathogens and near neighbor species as well as identifying mutant or engineered bio-threats (e.g., the use of pathogens for biological warfare or terrorism). The biodetection methods and compositions described herein can be used in a wide variety of applications such as, e.g., blood product surveillance, agriculture, forensic microbiology, epidemiology, food safety and bio-defense. The biodetection methods and compositions described herein make use of molecular inversion probes having recognition sequences at each terminus that hybridize to a matching target DNA sequence. The capture event is achieved by polymerase driven extension from the 3′ end of the capture probe to copy the target, followed by ligation to the 5′ end to complete the circle. The resulting circularized probes are enriched (e.g., via rolling circular amplification) and sequenced. This multiplexed reaction followed by sequencing allows for the detection of over 75,000 target gene regions in a single reaction.

In certain exemplary embodiments, molecular inversion probes (MIPs) useful in the biodetection methods described herein are designed to target nucleic acid sequences (e.g., housekeeping genes or regions of interest within a gene that are known to code for a protein that has been shown to be unique to the pathogenic strain either in its wild type or mutant form). Such target nucleic acid sequences (e.g., genes) can govern virulence, drug resistance and/or contain single nucleotide polymorphisms (SNPs) that discriminate between virulent pathogens and “near neighbor” species. These target nucleic acid sequences (e.g., housekeeping genes) are detectible in a mixture containing similar non-virulent strains and are resistant to gene transfer. It has been determined that any engineered or mutant forms of the subject pathogen can be identifiable via sequencing of this gene of interest.

The methods and compositions described herein are particularly useful in the infectious disease diagnostic field including, but not limited to, molecular diagnostic products, immunoassays, culture products and the like. Target markets include, but are not limited to, hospital bio-surveillance (e.g., HAI and other infectious diseases); hospital, clinical and reference lab diagnostics; public health agencies (e.g., CDC and DHS) with respect to the detection and identification of allergens, viruses, fungi, emerging infectious diseases and the like; food microbiology. Pathogenic targets include validated and potential biological weapons, validated bio-crime agents, globally and/or medically relevant animal pathogens, globally and/or medically relevant plant pathogens, organism having a high potential for bioengineering, zoonotic agents, toxins, foodborne pathogens, emerging infectious agents and the like. Specific pathogenic targets further include, but are not limited to Category A, B, C Priority pathogens (National institutes of Allergy and Infectious Disease), Select Agents (Health and Human Services), High Consequence plant and animal pathogens (U.S. Department of Agriculture), Notifiable Agents (Centers for Disease Control) and the like, as well as other pathogens described further herein.

The methods and compositions described herein are also broadly useful as diagnostic tools for monitoring a variety of disorders and/or diseases in a subject. In a non-limiting example, a database including specific cancer markers could be used to design MIPs to diagnose, prognose and/or monitor the presence of cancer in a sample (e.g., a biological sample).

The biodetection methods described herein include the steps of sample collection, library preparation, MIP hybridization and subsequent capture, amplification, and sequencing.

Samples or specimens containing target polynucleotides, such as fragments of genomic DNA and/or genomic RNA, may be collected from a wide variety of sources for use with the present invention, including, but not limited to, cell cultures, animal or plant tissues, patient biopsies, environmental samples, and the like. Samples are prepared using conventional techniques, which typically depend on the source from which a sample or specimen is taken.

As used herein, the term “sample” refers to a quantity of material from a biological, environmental, medical, or patient source in which detection or measurement of target nucleic acids is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool or tissue), as well as liquid and solid food and feed products and ingredients such as dairy items, vegetables, meat and meat by-products, and waste. Biological samples may include materials taken from a patient including, but not limited to cultures, cells, tissues, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

Prior to carrying out reactions on a sample, it will often be desirable to perform one or more sample preparation operations upon the sample. Typically, these sample preparation operations will include such manipulations as extraction of intracellular material, e.g., nucleic acids from whole cell samples, viruses and the like.

In considering genetic signatures suitable for the discriminatory identification of pathogenic strains from close relatives, a pathogen polymorphism database has been compiled based on gene regions that code for drug resistance, antigens, toxins and surface protein. These regions of interest have also been shown to be specific to target pathogens in environmental samples. Genes or regions of interest within a gene that has been shown to code for phenotypic traits such as, e.g., drug resistance, virulence, toxins, surface proteins and the like that have also been shown to be unique to the pathogen either in its wild-type or mutant forms were selected. The following criteria, which provided quality control, were used in the evaluation of candidate genes before addition to the database: 1) Genes references in published studies of clinical and environmental pathogen isolates; 2) The regions of interest occur in isolates that have been characterized by phenotypic drug sensitivity testing; 3) The regions of interest could be identified by specification of the gene nucleotide position and the nucleotide or amino acid change; and 4) a BLAST evaluation was carried out to verify uniqueness of gene of interest. MIP probes were designed to capture an entire gene or region of interest within a gene that includes a region of interest (such as, e.g., a SNP, a resistance mutation or the like) using software such as MIPTAG Pro. Nucleotide position and a set of genomes were input. MIP probes were generated using these criteria. It is to be understood, however, that additional databases could be designed using methods and reagents that are well-known in the art to adapt various known biomarkers for a variety of purposes (e.g., disease diagnosing, disease prognosing, disease monitoring and the like).

As used herein, “MIP technology” refers to a high-throughput genotyping technology capable of interrogating nucleic acid sequence of interest (e.g., single base changes, single nucleotide polymorphisms, drug resistance mutation and the like) on a large scale. Methods of using molecular inversion probe technology in highly multiplexed genotyping of SNPs are known. See Hardenbol et al. Genome Res. (2005) 15:269 and Hardenbol et al. (2003) Nat. Biotechnol. 21:673. The use of molecular inversion probe technology in allele quantification is also known. See Wang et al. (2005) Nucl. Acids Res. 33(21).

Generally, MIP technology is directed to the use of an oligonucleotide probe with recognition sequences at each terminus and optionally, one or more barcode sequences. The probe is hybridized with a target (e.g., genomic) nucleic acid sequence such that it forms a circular structure, with the ends of the probe abutting. This leaves a single base gap at the location of a nucleic acid sequence of interest. Probe designs having a gap length greater than one base are also useful in assays in which it is desirable to capture longer target DNA sequences. This gapped-duplex is then tested in four separate reactions, each with a single dNTP species present, in which successful polymerization and ligation provides allelic differentiation. The probes are subsequently released from the target nucleic acid sequence and those that have been covalently circularized in the correct allele/nucleotide reaction combination are amplified using a “universal” PCR primer pair. Tags are selected to have a similar Tₘ and base composition and to be maximally orthogonal in sequence complementarity.

According to aspects of the present invention, molecular inversion probes are used based on the methods described in Hardenbol, Nature Biotech., Vol. 21, No. 6., 6 Jun. 1993, Hardenbol et al., Genome Research, 2005; 15(2):269-75; Fakhrai et al. (2003) Nature Biotech. 21(6):673 and Wang et al. (2005) Nucl. Acids Res. 33:e183. The probe includes two regions of homology to a target nucleic acid sequence (e.g., a SNP, an antibiotic resistance gene or the like) located at the termini or end of the probe and two PCR primer regions common to all probes, and two common cleavage sites. A universal detection tag sequence can optionally be included for array detection of amplified probe. Cleavage sites are used to release the circularized probe from a target nucleic acid sequence and for post-amplification processing.

According to one aspect of the present invention, methods are provided whereby shareable probe pools are used. According to this aspect, large quantities and diverse numbers of MIP probes are generated on oligonucleotide chips (e.g. Agilent) in a way that is poolable & amplifiable (and hence easily shared). Each MIP oligo is flanked by universal oligos for amplification which can be removed. The following approaches are used to isolate the appropriate strand of the double stranded PCR products as well as to remove the universal primer regions mentioned above. (1) using one or more 3′ phosphothioate nucleotides on one of the two primers, (2) using exonucleases sensitive to 3′ or 5′ overhang (or lack thereof). One primer has one or more dU and can be removed by USER (which is a mixture of uracil DNA glycosylase and DNA glycosylase-lyase Endonuclease VIII) then the other primer has rU which can be cleaved by alkali. (3) using solid phase immobilization (e.g. magnetic bead streptavidin) of one primer with selective release of the other strand using alkali or heat to melt the base-pairs. (4) using asymmetric PCR (using an excess of the desired strand's primer) and (5) using separation by size and/or electrophoretic differences of the two strands by engineering the oligos to have different lengths (either by use of the rU or dU methods or 2′O methyl groups to block PCR extension beyond the 2′Ome.

According to certain aspects of the present invention, molecular inversion probes can be manufactured having gaps larger than one nucleotide and without extending the length of the molecular inversion probe. According to one aspect, the single stranded regions of the MIP during ligation reaction are free to extend far beyond the usual 0.34 nm/base and are free to rotate, unlike perfect CCC. Alternatively, very small DNA circles can be made according to the methods described in Bates et al. (1989) EMBO J. 8:1861. According to the present invention, smaller MIP probes aimed at large targets are believed to perform better in the range of 300 to 900 base pairs, which is advantageous for exons and other conserved elements.

In certain exemplary embodiments, a mixture of sample nucleic acid sequences, a plurality of probes and thermostable ligase and polymerase is heat denatured and brought to annealing temperature. Two sequences targeting each terminus of the probe hybridize to complementary sites in the target nucleic acid sequence, creating a circular conformation with a single-nucleotide gap between the termini of the probe. According to an alternate embodiment, the gap may be greater than one nucleotide. The genomic DNA is then split into four separate samples. Unlabeled dATP, dCTP, dGTP or dTTP is added to each of four samples. In reactions where the added nucleotide is complementary to the single base gap, DNA polymerase adds the nucleotide and DNA ligase closes the gap to form a covalently closed circular molecule that encircles the genomic strand to which it is hybridized. Exonucleases are added to digest linear probes in reactions where the added nucleotide was not complementary to the gap and excess linear probe in reactions where circular molecules were formed. The reactions are then heated to inactivate the exonucleases. To release probes from sample nucleic acid sequences, uracil-N-glycosylase is added to depurinate the uracil residues in the probes. The mixture is then heated to cleave the molecule at the abasic site and release it from sample nucleic acid sequences. Alternatively, the molecule can be removed from the sample nucleic acid sequences through methods other than cleavage, thereby leaving the molecule in its circular form.

Amplification can then be performed. Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, isothermal amplification (e.g., rolling circle amplification (RCA), hyperbranched rolling circle amplification (HRCA), strand displacement amplification (SDA), helicase-dependent amplification (HDA), PWGA) or any other nucleic acid amplification method using techniques well known to those of skill in the art. polymerase and/or ligase chain reactions. thermal cycling (PCR) or isothermally (e.g. RCA, hRCA, SDA, HDA, PWGA (Worldwide Website: biohelix.com/technology.asp)).

Several suitable RCA methods are known in the art. For example, linear RCA amplifies circular DNA by polymerase extension of a complementary primer. This process generates concatemerized copies of the circular DNA template such that multiple copies of a DNA sequence arranged end to end in tandem are generated. Exponential RCA is similar to the linear process except that it uses a second primer of identical sequence to the DNA circle (Lizardi et al. (1998) Nat. Genet. 19:225). This two-primer system achieves isothermal, exponential amplification. Exponential RCA has been applied to the amplification of non-circular DNA through the use of a linear probe that binds at both of its ends to contiguous regions of a target DNA followed by circularization using DNA ligase (i.e., padlock RCA) (Nilsson et al. (1994) Science 265(5181):2085). Hyperbranched RCA uses a second primer complementary to the rolling circle replication (RCR) product. This allows RCR products to be replicated by a strand-displacement mechanism, which can yield a billion-fold amplification in an isothermal reaction (Dahl et al. (2004) Proc. Natl. Acad. Sci. U.S.A. 101(13):4548).

“Polymerase chain reaction,” or “PCR,” refers to a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature greater than 90° C., primers annealed at a temperature in the range 50-75° C., and primers extended at a temperature in the range 72-78° C.

The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g., 200 nL, to a few hundred microliters, e.g., 200 microliters. “Reverse transcription PCR,” or “RT-PCR,” means a PCR that is preceded by a reverse transcription reaction that converts a target RNA to a complementary single stranded DNA, which is then amplified, e.g., Tecott et al., U.S. Pat. No. 5,168,038. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al., U.S. Pat. No. 5,210,015 (“Taqman”); Wittwer et al., U.S. Pat. Nos. 6,174,670 and 6,569,627 (intercalating dyes); Tyagi et al., U.S. Pat. No. 5,925,517 (molecular beacons). Detection chemistries for real-time PCR are reviewed in Mackay et al., Nucleic Acids Research, 30:1292-1305 (2002). “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al. (1999) Anal. Biochem., 273:221-228 (two-color real-time PCR). Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Techniques for quantitative PCR are well-known to those of ordinary skill in the art, as exemplified in the following references: Freeman et al., Biotechniques, 26:112-126 (1999); Becker-Andre et al., Nucleic Acids Research, 17:9437-9447 (1989); Zimmerman et al., Biotechniques, 21:268-279 (1996); Diviacco et al., Gene, 122:3013-3020 (1992); Becker-Andre et al., Nucleic Acids Research, 17:9437-9446 (1989); and the like.

In certain embodiments, methods of determining the nucleic acid sequence of one or more nucleic acid sequences (e.g., target nucleic acid sequences) are provided. Determination of a target nucleic acid sequence can be performed using variety of sequencing methods known in the art including, but not limited to, sequencing by hybridization (SBH), sequencing by ligation (SBL), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., on cyclic array sequencing using platforms such as Roche 454, Illumina Solexa, AB-SOLiD, Helicos, Polonator platforms and the like, can also be utilized. High-throughput sequencing methods are described in U.S. Ser. No. 61/162,913, filed Mar. 24, 2009. A variety of light-based sequencing technologies are known in the art (Landegren et al. (1998) Genome Res. 8:769-76; Kwok (2000) Pharmocogenomics 1:95-100; and Shi (2001) Clin. Chem. 47:164-172).

In certain exemplary embodiments, methods for identifying the presence or absence of a pathogen are provided. As used herein, the term “pathogen” includes, but is not limited to, a pathogenic organism such as a virus, a bacterium, a fungus, a parasite, an infectious protein and the like.

Viruses include, but are not limited to, DNA or RNA animal viruses. As used herein, RNA viruses include, but are not limited to, virus families such as Picornaviridae (e.g., polioviruses), Reoviridae (e.g., rotaviruses), Togaviridae (e.g., encephalitis viruses, yellow fever virus, rubella virus), Orthomyxoviridae (e.g., influenza viruses), Paramyxoviridae (e.g., respiratory syncytial virus, measles virus, mumps virus, parainfluenza virus), Rhabdoviridae (e.g., rabies virus), Coronaviridae, Bunyaviridae, Flaviviridae, Filoviridae, Arenaviridae, Bunyaviridae and Retroviridae (e.g., human T cell lymphotropic viruses (HTLV), human immunodeficiency viruses (HIV)). As used herein, DNA viruses include, but are not limited to, virus families such as Papovaviridae (e.g., papilloma viruses), Adenoviridae (e.g., adenovirus), Herpesviridae (e.g., herpes simplex viruses), and Poxviridae (e.g., variola viruses).

Bacteria include, but are not limited to, gram positive bacteria, gram negative bacteria, acid-fast bacteria and the like.

As used herein, gram positive bacteria include, but are not limited to, Actinomedurae, Actinomyces israelii, Bacillus anthracis, Bacillus cereus, Clostridium botulinum, Clostridium difficile, Clostridium perfringens, Clostridium tetani, Corynebacterium, Enterococcus faecalis, Listeria monocytogenes, Nocardia, Propionibacterium acnes, Staphylococcus aureus, Staphylococcus epiderm, Streptococcus mutans, Streptococcus pneumoniae and the like.

As used herein, gram negative bacteria include, but are not limited to, Afipia felis, Bacteriodes, Bartonella bacilliformis, Bortadella pertussis, Borrelia burgdorferi, Borrelia recurrentis, Brucella, Calymmatobacterium granulomatis, Campylobacter, Escherichia coli, Francisella tularensis, Gardnerella vaginalis, Haemophilius aegyptius, Haemophilius ducreyi, Haemophilius influenziae, Heliobacter pylori, Legionella pneumophila, Leptospira interrogans, Neisseria meningitidia, Porphyromonas gingivalis, Providencia sturti, Pseudomonas aeruginosa, Salmonella enteridis, Salmonella typhi, Serratia marcescens, Shigella boydii, Streptobacillus moniliformis, Streptococcus pyogenes, Treponema pallidum, Vibrio cholerae, Yersinia enterocolitica, Yersinia pestis and the like.

As used herein, acid-fast bacteria include, but are not limited to, Myobacterium avium, Myobacterium leprae, Myobacterium tuberculosis and the like.

As used herein, other bacteria not falling into the other three categories include, but are not limited to, Bartonella henseiae, Chlamydia psittaci, Chlamydia trachomatis, Coxiella burnetii, Mycoplasma pneumoniae, Rickettsia akari, Rickettsia prowazekii, Rickettsia rickettsii, Rickettsia tsutsugamushi, Rickettsia typhi, Ureaplasma urealyticum, Diplococcus pneumoniae, Ehrlichia chafensis, Enterococcus faecium, Meningococci and the like.

As used herein, fungi include, but are not limited to, Aspergilli, Candidae, Candida albicans, Coccidioides immitis, Cryptococci, and combinations thereof.

As used herein, parasitic microbes include, but are not limited to, Balantidium coli, Cryptosporidium parvum, Cyclospora cayatanensis, Encephalitozoa, Entamoeba histolytica, Enterocytozoon bieneusi, Giardia lamblia, Leishmaniae, Plasmodii, Toxoplasma gondii, Trypanosomae, trapezoidal amoeba and the like.

As used herein, parasites include worms (e.g., helminthes), particularly parasitic worms including, but not limited to, Nematoda (roundworms, e.g., whipworms, hookworms, pinworms, ascarids, filarids and the like), Cestoda (e.g., tapeworms)

As used herein, infectious proteins include prions. Disorders caused by prions include, but are not limited to, human disorders such as Creutzfeldt-Jakob disease (CJD) (including, e.g., iatrogenic Creutzfeldt-Jakob disease (iCJD), variant Creutzfeldt-Jakob disease (vCJD), familial Creutzfeldt-Jakob disease (fCJD), and sporadic Creutzfeldt-Jakob disease (sCJD)), Gerstmann-Sträussler-Scheinker syndrome (GSS), fatal familial insomnia (fFI), sporadic fatal insomnia (sFI), kuru, and the like, as well as disorders in animals such as scrapie (sheep and goats), bovine spongiform encephalopathy (BSE) (cattle), transmissible mink encephalopathy (TME) (mink), chronic wasting disease (CWD) (elk, mule deer), feline spongiform encephalopathy (cats), exotic ungulate encephalopathy (EUE) (nyala, oryx, greater kudu), spongiform encephalopathy of the ostrich and the like.

In certain exemplary embodiments, methods of prognosing, diagnosing and/or monitoring one or more cellular proliferative disorders are provided. Cellular proliferative disorders are intended to include disorders associated with rapid proliferation. As used herein, the term “cellular proliferative disorder” includes disorders characterized by undesirable or inappropriate proliferation of one or more subset(s) of cells in a multicellular organism. The term “cancer” refers to various types of malignant neoplasms, most of which can invade surrounding tissues, and may metastasize to different sites (see, for example, PDR Medical Dictionary 1st edition (1995), incorporated herein by reference in its entirety for all purposes). The terms “neoplasm” and “tumor” refer to an abnormal tissue that grows by cellular proliferation more rapidly than normal. Id. Such abnormal tissue shows partial or complete lack of structural organization and functional coordination with the normal tissue which may be either benign (i.e., benign tumor) or malignant (i.e., malignant tumor).

Examples of the types of neoplasms intended to be encompassed by the present invention include but are not limited to those neoplasms associated with cancers of neural tissue, blood forming tissue, breast, skin, bone, prostate, ovaries, uterus, cervix, liver, lung, brain, larynx, gallbladder, pancreas, rectum, parathyroid, thyroid, adrenal gland, immune system, head and neck, colon, stomach, bronchi, and/or kidneys.

For those embodiments where whole cells, viruses or other tissue samples are being analyzed, it will typically be necessary to extract the nucleic acids from the cells or viruses, prior to continuing with the various sample preparation operations. Accordingly, following sample collection, nucleic acids may be liberated from the collected cells, viral coat, etc., into a crude extract, followed by additional treatments to prepare the sample for subsequent operations, e.g., denaturation of contaminating (DNA binding) proteins, purification, filtration, desalting, and the like. Liberation of nucleic acids from the sample cells or viruses, and denaturation of DNA binding proteins may generally be performed by chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within a sample preparation chamber, a separate accessible chamber, or may be externally introduced.

Following extraction, it will often be desirable to separate the nucleic acids from other elements of the crude extract, e.g., denatured proteins, cell membrane particles, salts, and the like. Removal of particulate matter is generally accomplished by filtration, flocculation or the like. A variety of filter types may be readily incorporated into the device. Further, where chemical denaturing methods are used, it may be desirable to desalt the sample prior to proceeding to the next step. Desalting of the sample, and isolation of the nucleic acid may generally be carried out in a single step, e.g., by binding the nucleic acids to a solid phase and washing away the contaminating salts or performing gel filtration chromatography on the sample, passing salts through dialysis membranes, and the like. Suitable solid supports for nucleic acid binding include, e.g., diatomaceous earth, silica (i.e., glass wool), or the like. Suitable gel exclusion media, also well known in the art, may also be readily incorporated into the devices of the present invention, and is commercially available from, e.g., Pharmacia and Sigma Chemical.

In some applications, such as measuring target polynucleotides in rare cells from a patient's blood, an enrichment step may be carried out prior to conducting an assay, such as by immunomagnetic isolation, fluorescent cell sorting or other such technique. Such isolation or enrichment may be carried out using a variety of techniques and materials known in the art, as disclosed in the following representative references: Terstappen et al., U.S. Pat. No. 6,365,362; Terstappen et al., U.S. Pat. No. 5,646,001; Rohr et al., U.S. Pat. No. 5,998,224; Kausch et al., U.S. Pat. No. 5,665,582; Kresse et al., U.S. Pat. No. 6,048,515; Kausch et al., U.S. Pat. No. 5,508,164; Miltenyi et al., U.S. Pat. No. 5,691,208; Molday, U.S. Pat. No. 4,452,773; Kronick, U.S. Pat. No. 4,375,407; Radbruch et al., Chapter 23, in Methods in Cell Biology, Vol. 42 (Academic Press, New York, 1994); Uhlen et al., Advances in Biomagnetic Separation (Eaton Publishing, Natick, 1994); Safarik et al., J. Chromatography B, 722: 33-53 (1999); Miltenyi et al., Cytometry, 11: 231-238 (1990); Nakamura et al., Biotechnol. Prog., 17: 1145-1155 (2001); Moreno et al., Urology, 58: 386-392 (2001); Racila et al., Proc. Natl. Acad. Sci., 95: 4589-4594 (1998); Zigeuner et al., J. Urology, 169: 701-705 (2003); Ghossein et al., Seminars in Surgical Oncology, 20: 304-311 (2001).

Terms and symbols of nucleic acid chemistry, biochemistry, genetics, and molecular biology used herein follow those of standard treatises and texts in the field, e.g., Komberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See Kanehisa (1984) Nucl. Acids Res. 12:203. According to the present invention, useful MIP primer sequences hybridize to sequences that flank the nucleotide base or series of bases to be captured.

“Complex” means an assemblage or aggregate of molecules in direct or indirect contact with one another. In one aspect, “contact,” or more particularly, “direct contact,” in reference to a complex of molecules or in reference to specificity or specific binding, means two or more molecules are close enough so that attractive noncovalent interactions, such as van der Waal forces, hydrogen bonding, ionic and hydrophobic interactions, and the like, dominate the interaction of the molecules. In such an aspect, a complex of molecules is stable in that under assay conditions the complex is thermodynamically more favorable than a non-aggregated, or non-complexed, state of its component molecules. As used herein, “complex” refers to a duplex or triplex of polynucleotides or a stable aggregate of two or more proteins. In regard to the latter, a complex is formed by an antibody specifically binding to its corresponding antigen.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. In one aspect, stable duplex means that a duplex structure is not destroyed by a stringent wash, e.g., conditions including temperature of about 5° C. less that the Tₘ of a strand of the duplex and low monovalent salt concentration, e.g., less than 0.2 M, or less than 0.1 M. “Perfectly matched” in reference to a duplex means that the polynucleotide or oligonucleotide strands making up the duplex form a double stranded structure with one another such that every nucleotide in each strand undergoes Watson-Crick base pairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Genetic locus,” or “locus” in reference to a genome or target polynucleotide, means a contiguous subregion or segment of the genome or target polynucleotide. As used herein, genetic locus, or locus, may refer to the position of a nucleotide, a gene, or a portion of a gene in a genome, including mitochondrial DNA, or it may refer to any contiguous portion of genomic sequence whether or not it is within, or associated with, a gene. In one aspect, a genetic locus refers to any portion of genomic sequence, including mitochondrial DNA, from a single nucleotide to a segment of few hundred nucleotides, e.g. 100-300, in length. Usually, a particular genetic locus may be identified by its nucleotide sequence, or the nucleotide sequence, or sequences, of one or both adjacent or flanking regions. In another aspect, a genetic locus refers to the expressed nucleic acid product of a gene, such as an RNA molecule or a cDNA copy thereof.

“Hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid” or “duplex.” “Hybridization conditions” will typically include salt concentrations of less than about 1 M, more usually less than about 500 mM and even more usually less than about 200 mM. Hybridization temperatures can be as low as 5° C., but are typically greater than 22° C., more typically greater than about 30° C., and often in excess of about 37° C. Hybridizations are usually performed under stringent conditions, i.e., conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the Tₘ for the specific sequence at s defined ionic strength and pH. Exemplary stringent conditions include salt concentration of at least 0.01 M to no more than 1 M Na ion concentration (or other salts) at a pH 7.0 to 8.3 and a temperature of at least 25° C. For example, conditions of 5× SSPE (750 mM NaCl, 50 mM Na phosphate, 5 mM EDTA, pH 7.4) and a temperature of 25-30° C. are suitable for allele-specific probe hybridizations. For stringent conditions, see for example, Sambrook, Fritsche and Maniatis, Molecular Cloning A Laboratory Manual, 2nd Ed. Cold Spring Harbor Press (1989) and Anderson Nucleic Acid Hybridization, 1^{st} Ed., BIOS Scientific Publishers Limited (1999). “Hybridizing specifically to” or “specifically hybridizing to” or like expressions refer to the binding, duplexing, or hybridizing of a molecule substantially to or only to a particular nucleotide sequence or sequences under stringent conditions when that sequence is present in a complex mixture (e.g., total cellular) DNA or RNA.

“Hybridization-based assay” means any assay that relies on the formation of a stable complex as the result of a specific binding event. In one aspect, a hybridization-based assay means any assay that relies on the formation of a stable duplex or triplex between a probe and a target nucleotide sequence for detecting or measuring such a sequence. In one aspect, probes of such assays anneal to (or form duplexes with) regions of target sequences in the range of from 8 to 100 nucleotides; or in other aspects, they anneal to target sequences in the range of from 8 to 40 nucleotides, or more usually, in the range of from 8 to 20 nucleotides. A “probe” in reference to a hybridization-based assay means a polynucleotide that has a sequence that is capable of forming a stable hybrid (or triplex) with its complement in a target nucleic acid and that is capable of being detected, either directly or indirectly. Hybridization-based assays include, without limitation, assays that use the specific base-pairing of one or more oligonucleotides as target recognition components, such as polymerase chain reactions, NASBA reactions, oligonucleotide ligation reactions, single-base extension reactions, circularizable probe reactions, allele-specific oligonucleotide hybridizations, either in solution phase or bound to solid phase supports, such as microarrays or microbeads, and the like. An important subset of hybridization-based assays include such assays that have at least one enzymatic processing step after a hybridization step. Hybridization-based assays of this subset include, without limitation, polymerase chain reactions, NASBA reactions, oligonucleotide ligation reactions, cleavase reactions, e.g., in INVADER™ assays, single-base extension reactions, probe circularization reactions, and the like. There is extensive guidance in the literature on hybridization-based assays, e.g., Hames et al., editors, Nucleic Acid Hybridization a Practical Approach (IRL Press, Oxford, 1985); Tijssen, Hybridization with Nucleic Acid Probes, Parts I & II (Elsevier Publishing Company, 1993); Hardiman, Microarray Methods and Applications (DNA Press, 2003); Schena, editor, DNA Microarrays a Practical Approach (IRL Press, Oxford, 1999); and the like. In one aspect, hybridization-based assays are solution phase assays; that is, both probes and target sequences hybridize under conditions that are substantially free of surface effects or influences on reaction rate. A solution phase assay includes circumstances where either probes or target sequences are attached to microbeads such that the attached sequences have substantially the same environment (e.g., permitting reagent access, etc.) as free sequences. In another aspect, hybridization-based assays include immunoassays wherein antibodies employ nucleic acid reporters based on amplification. In such assays, antibody probes specifically bind to target molecules, such as proteins, in separate reactions, after which the products of such reactions (i.e., antibody-protein complexes) are combined and nucleic acid reporters are amplified. Preferably, such nucleic acid reporters include oligonucleotide tags that are converted enzymatically into labeled oligonucleotide tags for analysis on a microarray, as described below. The following exemplary references disclose antibody-nucleic acid conjugates for immunoassays: Baez et al., U.S. Pat. No. 6,511,809; Sano et al., U.S. Pat. No. 5,665,539; Eberwine et al., U.S. Pat. No. 5,922,553; Landegren et al., U.S. Pat. No. 6,558,928; Landegren et al., U.S. Patent Pub. 2002/0064779; and the like. In particular, the two latter patent publications by Landegren et al. disclose steps of forming amplifiable probes after a specific binding event.

“Kit” refers to any delivery system for delivering materials or reagents for carrying out a method of the invention. In the context of assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials for assays of the invention. Such contents may be delivered to the intended recipient together or separately. For example, a first container may contain an enzyme for use in an assay, while a second container contains probes.

“Ligation” means to form a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references: Whitely et al., U.S. Pat. No. 4,883,750; Letsinger et al., U.S. Pat. No. 5,476,930; Fung et al., U.S. Pat. No. 5,593,826; Kool, U.S. Pat. No. 5,426,180; Landegren et al., U.S. Pat. No. 5,871,921; Xu and Kool (1999) Nucl. Acids Res. 27:875; Higgins et al., Meth. in Enzymol. (1979) 68:50; Engler et al. (1982) The Enzymes, 15:3 (1982); and Namsaraev, U.S. Patent Pub. 2004/0110213.

“Microarray” refers in one embodiment to a type of multiplex assay product that comprises a solid phase support having a substantially planar surface on which there is an array of spatially defined non-overlapping regions or sites that each contain an immobilized hybridization probe. “Substantially planar” means that features or objects of interest, such as probe sites, on a surface may occupy a volume that extends above or below a surface and whose dimensions are small relative to the dimensions of the surface. For example, beads disposed on the face of a fiber optic bundle create a substantially planar surface of probe sites, or oligonucleotides disposed or synthesized on a porous planar substrate creates a substantially planar surface. Spatially defined sites may additionally be “addressable” in that its location and the identity of the immobilized probe at that location are known or determinable. Probes immobilized on microarrays include nucleic acids, such as oligonucleotide barcodes, that are generated in or from an assay reaction. Typically, the oligonucleotides or polynucleotides on microarrays are single stranded and are covalently attached to the solid phase support, usually by a 5′-end or a 3′-end. The density of non-overlapping regions containing nucleic acids in a microarray is typically greater than 100 per cm², and more preferably, greater than 1000 per cm². Microarray technology relating to nucleic acid probes is reviewed in the following exemplary references: Schena, Editor, Microarrays: A Practical Approach (IRL Press, Oxford, 2000); Southern, Current Opin. Chem. Biol., 2: 404-410 (1998); Nature Genetics Supplement, 21:1-60 (1999); and Fodor et al., U.S. Pat. Nos. 5,424,186; 5,445,934; and 5,744,305. A microarray may comprise arrays of microbeads, or other microparticles, alone or disposed on a planar surface or in wells or other physical configurations that can be sued to separate the beads. Such microarrays may be formed in a variety of ways, as disclosed in the following exemplary references: Brenner et al. (2000) Nat. Biotechnol. 18:630; Tulley et al., U.S. Pat. No. 6,133,043; Stuelpnagel et al., U.S. Pat. No. 6,396,995; Chee et al., U.S. Pat. No. 6,544,732; and the like. In one format, microarrays are formed by randomly disposing microbeads having attached oligonucleotides on a surface followed by determination of which microbead carries which oligonucleotide by a decoding procedure, e.g. as disclosed by Gunderson et al., U.S. Patent Pub. No. 2003/0096239.

“Microarrays” or “arrays” can also refer to a heterogeneous pool of nucleic acid molecules that is distributed over a support matrix. The nucleic acids can be covalently or noncovalently attached to the support. Preferably, the nucleic acid molecules are spaced at a distance from one another sufficient to permit the identification of discrete features of the array. Nucleic acids on the array may be non-overlapping or partially overlapping. Methods of transferring a nucleic acid pool to support media is described in U.S. Pat. No. 6,432,360. Bead based methods useful in the present invention are disclosed in PCT US05/04373.

“Amplifying” includes the production of copies of a nucleic acid molecule of the array or a nucleic acid molecule bound to a bead via repeated rounds of primed enzymatic synthesis. “In situ” amplification indicated that the amplification takes place with the template nucleic acid molecule positioned on a support or a bead, rather than in solution. In situ amplification methods are described in U.S. Pat. No. 6,432,360.

“Support” can refer to a matrix upon which nucleic acid molecules of a nucleic acid array are placed. The support can be solid or semi-solid or a gel. “Semi-solid” refers to a compressible matrix with both a solid and a liquid component, wherein the liquid occupies pores, spaces or other interstices between the solid matrix elements. Semi-solid supports can be selected from polyacrylamide, cellulose, polyamide (nylon) and crossed linked agarose, dextran and polyethylene glycol.

“Randomly-patterned” or “random” refers to non-ordered, non-Cartesian distribution (in other words, not arranged at pre-determined points along the x- or y-axes of a grid or at defined “clock positions,” degrees or radii from the center of a radial pattern) of nucleic acid molecules over a support, that is not achieved through an intentional design (or program by which such design may be achieved) or by placement of individual nucleic acid features. Such a “randomly-patterned” or “random” array of nucleic acids may be achieved by dropping, spraying, plating or spreading a solution, emulsion, aerosol, vapor or dry preparation comprising a pool of nucleic acid molecules onto a support and allowing the nucleic acid molecules to settle onto the support without intervention in any manner to direct them to specific sites thereon. Arrays of the invention can be randomly patterned or random.

“Heterogeneous” refers to a population or collection of nucleic acid molecules that comprises a plurality of different sequences. According to one aspect, a heterogeneous pool of nucleic acid molecules results from a preparation of RNA or DNA from a cell which may be unfractionated or partially-fractionated.

“Nucleoside” as used herein includes the natural nucleosides, including 2′-deoxy and 2′-hydroxyl forms, e.g. as described in Komberg and Baker, DNA Replication, 2nd Ed. (Freeman, San Francisco, 1992). “Analogs” in reference to nucleosides includes synthetic nucleosides having modified base moieties and/or modified sugar moieties, e.g., described by Scheit, Nucleotide Analogs (John Wiley, New York, 1980); Uhlman and Peyman, Chemical Reviews, 90:543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al., Exp. Opin. Ther. Patents, 6: 855-870 (1996); Mesmaeker et al., Current Opinion in Structural Biology, 5:343-355 (1995); and the like. Exemplary types of polynucleotides that are capable of enhancing duplex stability include oligonucleotide phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Oligonucleotide” or “polynucleotide,” which are used synonymously, means a linear polymer of natural or modified nucleosidic monomers linked by phosphodiester bonds or analogs thereof The term “oligonucleotide” usually refers to a shorter polymer, e.g., comprising from about 3 to about 100 monomers, and the term “polynucleotide” usually refers to longer polymers, e.g., comprising from about 100 monomers to many thousands of monomers, e.g., 10,000 monomers, or more. Oligonucleotides comprising probes or primers usually have lengths in the range of from 12 to 60 nucleotides, and more usually, from 18 to 40 nucleotides. Oligonucleotides and polynucleotides may be natural or synthetic. Oligonucleotides and polynucleotides include deoxyribonucleosides, ribonucleosides, and non-natural analogs thereof, such as anomeric forms thereof, peptide nucleic acids (PNAs), and the like, provided that they are capable of specifically binding to a target genome by way of a regular pattern of monomer-to-monomer interactions, such as Watson-Crick type of base pairing, base stacking, Hoogsteen or reverse Hoogsteen types of base pairing, or the like.

Usually nucleosidic monomers are linked by phosphodiester bonds. Whenever an oligonucleotide is represented by a sequence of letters, such as “ATGCCTG,” it will be understood that the nucleotides are in 5′ to 3′ order from left to right and that “A” denotes deoxyadenosine, “C” denotes deoxycytidine, “G” denotes deoxyguanosine, “T” denotes deoxythymidine, and “U” denotes the ribonucleoside, uridine, unless otherwise noted. Usually oligonucleotides comprise the four natural deoxynucleotides; however, they may also comprise ribonucleosides or non-natural nucleotide analogs. It is clear to those skilled in the art when oligonucleotides having natural or non-natural nucleotides may be employed in methods and processes described herein. For example, where processing by an enzyme is called for, usually oligonucleotides consisting solely of natural nucleotides are required. Likewise, where an enzyme has specific oligonucleotide or polynucleotide substrate requirements for activity, e.g., single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al., Molecular Cloning, Second Edition (Cold Spring Harbor Laboratory, New York, 1989), and like references. Oligonucleotides and polynucleotides may be single stranded or double stranded.

“Oligonucleotide tag” or “tag” means an oligonucleotide that is attached to a polynucleotide and is used to identify and/or track the polynucleotide in a reaction. Usually, an oligonucleotide tag is attached to the 3′- or 5′-end of a polynucleotide to form a linear conjugate, sometime referred to herein as a “tagged polynucleotide,” or equivalently, an “oligonucleotide tag-polynucleotide conjugate,” or “tag-polynucleotide conjugate.” Oligonucleotide tags may vary widely in size and compositions; the following references provide guidance for selecting sets of oligonucleotide tags appropriate for particular embodiments: Brenner, U.S. Pat. No. 5,635,400; Brenner et al., Proc. Natl. Acad. Sci., 97: 1665; Shoemaker et al. (1996) Nature Genetics, 14:450; Morris et al., EP Patent Pub. 0799897A1; Wallace, U.S. Pat. No. 5,981,179; and the like.

In one embodiment, an amplifiable probe of the invention comprises at least one oligonucleotide tag that is replicated and labeled to produce a labeled oligonucleotide probe. The nature of the label on an oligonucleotide tag can be based on a wide variety of physical or chemical properties including, but not limited to, light absorption, fluorescence, chemiluminescence, electrochemiluminescence, mass, charge, and the like. The signals based on such properties can be generated directly or indirectly. For example, a label can be a fluorescent molecule covalently attached to an amplified oligonucleotide tag that directly generates an optical signal. Alternatively, a label can comprise multiple components, such as a hapten-antibody complex, that, in turn, may include fluorescent dyes that generated optical signals, enzymes that generate products that produce optical signals, or the like. In certain aspects, the label on an oligonucleotide tag is a fluorescent label that is directly or indirectly attached to an amplified oligonucleotide tag.

Fluorescent labels and their attachment to oligonucleotides, such as oligonucleotide tags, are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); Wetmur, Critical Reviews in Biochemistry and Molecular Biology, 26:227-259 (1991); and the like. Particular methodologies applicable to the invention are disclosed in the following sample of references: Fung et al., U.S. Pat. No. 4,757,141; Hobbs, Jr., et al. U.S. Pat. No. 5,151,507; Cruickshank, U.S. Pat. No. 5,091,519. In one aspect, one or more fluorescent dyes are used as labels for labeled target sequences, e.g., as disclosed by Menchen et al., U.S. Pat. No. 5,188,934 (4,7-dichlorofluorscein dyes); Begot et al., U.S. Pat. No. 5,366,860 (spectrally resolvable rhodamine dyes); Lee et al., U.S. Pat. No. 5,847,162 (4,7-dichlororhodamine dyes); Khanna et al., U.S. Pat. No. 4,318,846 (ether-substituted fluorescein dyes); Lee et al., U.S. Pat. No. 5,800,996 (energy transfer dyes); Lee et al., U.S. Pat. No. 5,066,580 (xanthine dyes): Mathies et al., U.S. Pat. No. 5,688,648 (energy transfer dyes); and the like. Labelling can also be carried out with quantum dots, as disclosed in the following patents and patent publications: U.S. Pat. Nos. 6,322,901; 6,576,291; 6,423,551; 6,251,303; 6,319,426; 6,426,513; 6,444,143; 5,990,479; 6,207,392; 2002/0045045; 2003/0017264; and the like. As used herein, the term “fluorescent label” includes a signaling moiety that conveys information through the fluorescent absorption and/or emission properties of one or more molecules. Such fluorescent properties include fluorescence intensity, fluorescence life time, emission spectrum characteristics, energy transfer, and the like.

Commercially available fluorescent nucleotide analogues readily incorporated into the labeling oligonucleotides include, for example, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY MTR-14-dUTP, RHODAMINE GREEN™-5-dUTP, OREGON GREENR™ 488-5-dUTP, TEXAS RED™-12-dUTP, BODIPY TM 630/650-14-dUTP, BODIPY TM 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY TM FL-14-UTP, BODIPY TMR-14-UTP, BODIPY TM TR-14-UTP, RHODAMINE GREEN™-5-UTP, ALEXA FLUOR™ 488-5-UTP, LEXA FLUOR™ 546-14-UTP (Molecular Probes, Inc. Eugene, Oreg.). Protocols are available for custom synthesis of nucleotides having other fluorophores. Henegariu et al., “Custom Fluorescent-Nucleotide Synthesis as an Alternative Method for Nucleic Acid Labeling,” Nature Biotechnol. 18:345-348 (2000).

Other fluorophores available for post-synthetic attachment include, inter alia, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), and Cy2, Cy3.5, Cy5.5, and Cy7 (Amersham Biosciences, Piscataway, N.J. USA, and others).

FRET tandem fluorophores may also be used, such as PerCP-Cy5.5, PE-Cy5, PE-Cy5.5, PE-Cy7, PE-Texas Red, and APC-Cy7; also, PE-Alexa dyes (610, 647, 680) and APC-Alexa dyes.

Metallic silver particles may be coated onto the surface of the array to enhance signal from fluorescently labeled oligos bound to the array. Lakowicz et al. (2003) BioTechniques 34:62.

Biotin, or a derivative thereof, may also be used as a label on a detection oligonucleotide, and subsequently bound by a detectably labeled avidin/streptavidin derivative (e.g. phycoerythrin-conjugated streptavidin), or a detectably labeled anti-biotin antibody. Digoxigenin may be incorporated as a label and subsequently bound by a detectably labeled anti-digoxigenin antibody (e.g. fluoresceinated anti-digoxigenin). An aminoallyl-dUTP residue may be incorporated into a detection oligonucleotide and subsequently coupled to an N-hydroxy succinimide (NHS) derivatized fluorescent dye, such as those listed supra. In general, any member of a conjugate pair may be incorporated into a detection oligonucleotide provided that a detectably labeled conjugate partner can be bound to permit detection. As used herein, the term antibody refers to an antibody molecule of any class, or any sub-fragment thereof, such as an Fab.

Other suitable labels for detection oligonucleotides may include fluorescein (FAM), digoxigenin, dinitrophenol (DNP), dansyl, biotin, bromodeoxyuridine (BrdU), hexahistidine (6× His), phosphor-amino acids (e.g. P-tyr, P-ser, P-thr), or any other suitable label. In one embodiment the following hapten/antibody pairs are used for detection, in which each of the antibodies is derivatized with a detectable label: biotin/α-biotin, digoxigenin/a-digoxigenin, dinitrophenol (DNP)/α-DNP, 5-Carboxyfluorescein (FAM)/α-FAM.

As mentioned above, oligonucleotide tags can be indirectly labeled, especially with a hapten that is then bound by a capture agent, e.g., as disclosed in Holtke et al., U.S. Pat. Nos. 5,344,757; 5,702,888; and 5,354,657; Huber et al., U.S. Pat. No. 5,198,537; Miyoshi, U.S. Pat. No. 4,849,336; Misiura and Gait, PCT publication WO 91/17160; and the like. Many different hapten-capture agent pairs are available for use with the invention, either with a target sequence or with a detection oligonucleotide used with a target sequence, as described below. Exemplary, haptens include, biotin, des-biotin and other derivatives, dinitrophenol, dansyl, fluorescein, CY5, and other dyes, digoxigenin, and the like. For biotin, a capture agent may be avidin, streptavidin, or antibodies. Antibodies may be used as capture agents for the other haptens (many dye-antibody pairs being commercially available, e.g., Molecular Probes, Eugene, Oreg.).

“Polymorphism” or “genetic variant” means a substitution, inversion, insertion, or deletion of one or more nucleotides at a genetic locus, or a translocation of DNA from one genetic locus to another genetic locus. In one aspect, polymorphism means one of multiple alternative nucleotide sequences that may be present at a genetic locus of an individual and that may comprise a nucleotide substitution, insertion, or deletion with respect to other sequences at the same locus in the same individual, or other individuals within a population. An individual may be homozygous or heterozygous at a genetic locus; that is, an individual may have the same nucleotide sequence in both alleles, or have a different nucleotide sequence in each allele, respectively. In one aspect, insertions or deletions at a genetic locus comprises the addition or the absence of from 1 to 10 nucleotides at such locus, in comparison with the same locus in another individual of a population (or another allele in the same individual). Usually, insertions or deletions are with respect to a major allele at a locus within a population, e.g., an allele present in a population at a frequency of fifty percent or greater.

“Primer” includes an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of between 3 to 36 nucleotides, also 5 to 24 nucleotides, also from 14 to 36 nucleotides. Primers within the scope of the invention can be universal primers or non-universal primers. Pairs of primers can flank a sequence of interest or a set of sequences of interest. Primers and probes can be degenerate in sequence. Primers within the scope of the present invention bind adjacent to the target sequence, whether it is the sequence to be captured for analysis, or a tag that it to be copied.

“Solid support,” “support,” and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide. Semisolid supports and gel supports are also useful in the present invention, especially when polony amplification is used.

“Specific” or “specificity” in reference to the binding of one molecule to another molecule, such as a target sequence to a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak non-covalent chemical interactions, such as van der Waal forces, hydrogen bonding, base-stacking interactions, ionic and hydrophobic interactions, and the like, dominate the interaction of the molecules.

“Tₘ” is used in reference to “melting temperature.” Melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation. Tₘ=81.5+0.41 (% G+C), when a nucleic acid is in aqueous solution at 1 M NaCl (see e.g., Anderson and Young, “Quantitative Filter Hybridization,” in Nucleic Acid Hybridization (1985). Other references (e.g., Allawi, H. T. & Santa Lucia, J., Jr., Biochemistry 36, 10581-94 (1997)) include alternative methods of computation which take structural and environmental, as well as sequence characteristics into account for the calculation of Tₘ.

It is to be understood that the embodiments of the present invention which have been described are merely illustrative of some of the applications of the principles of the present invention. Numerous modifications may be made by those skilled in the art based upon the teachings presented herein without departing from the true spirit and scope of the invention. The contents of all references, patents and published patent applications cited throughout this application are hereby incorporated by reference in their entirety for all purposes.

The following examples are set forth as being representative of the present invention. These examples are not to be construed as limiting the scope of the invention as these and other equivalent embodiments will be apparent in view of the present disclosure, figures, tables, and accompanying claims.

### EXAMPLE I

### Two-Dimensional Genotyping

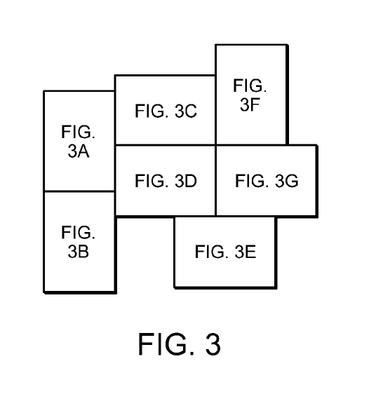

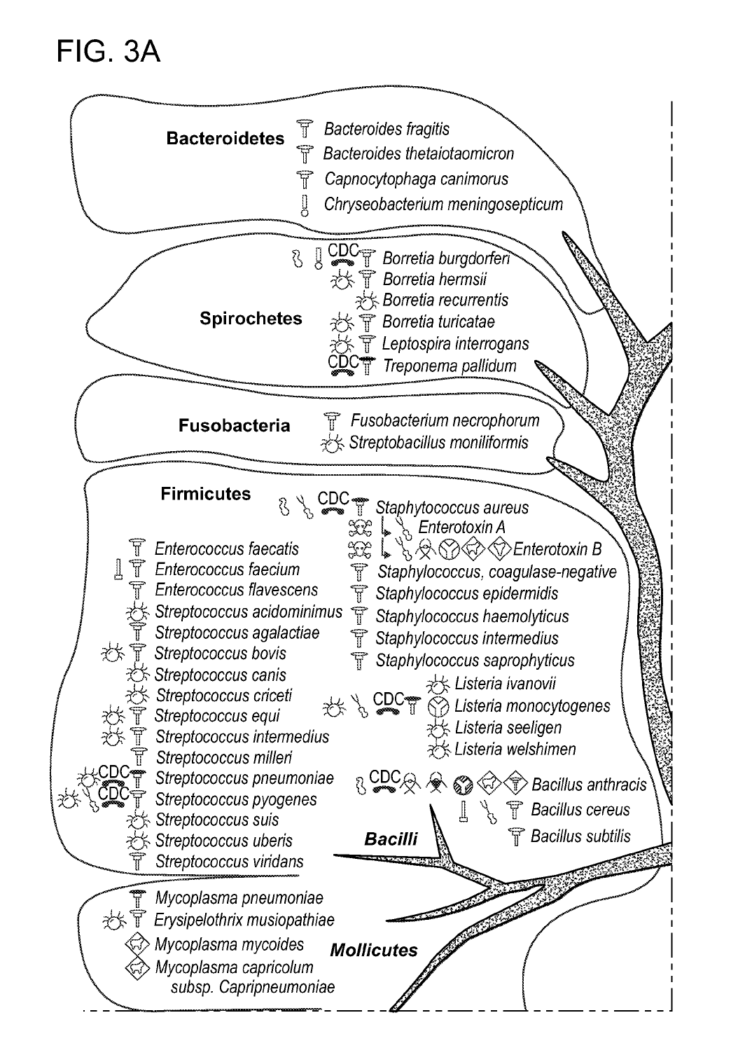

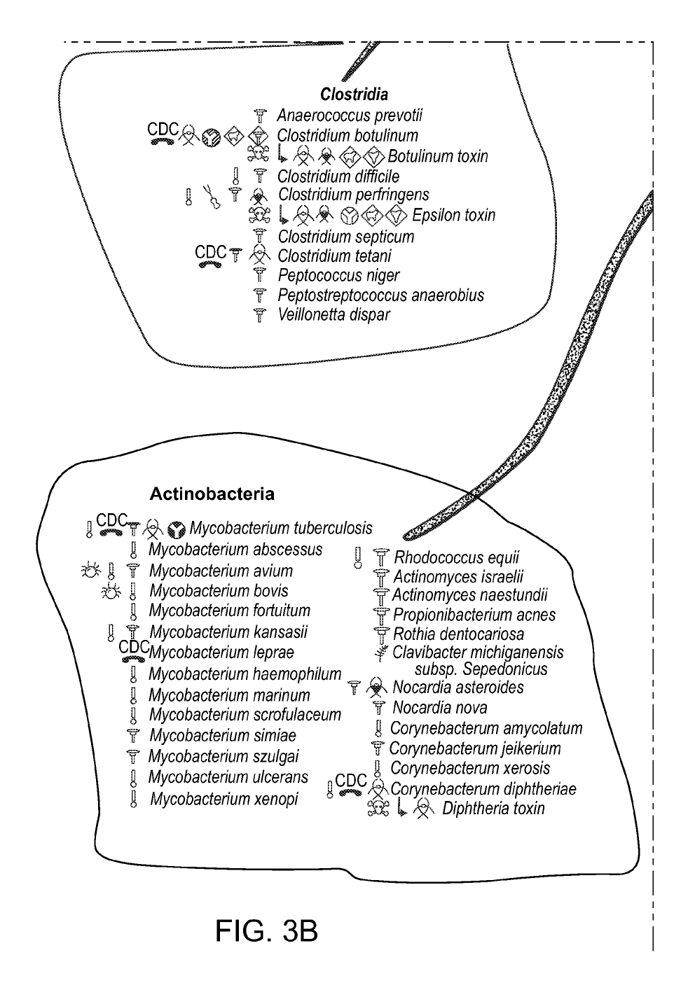

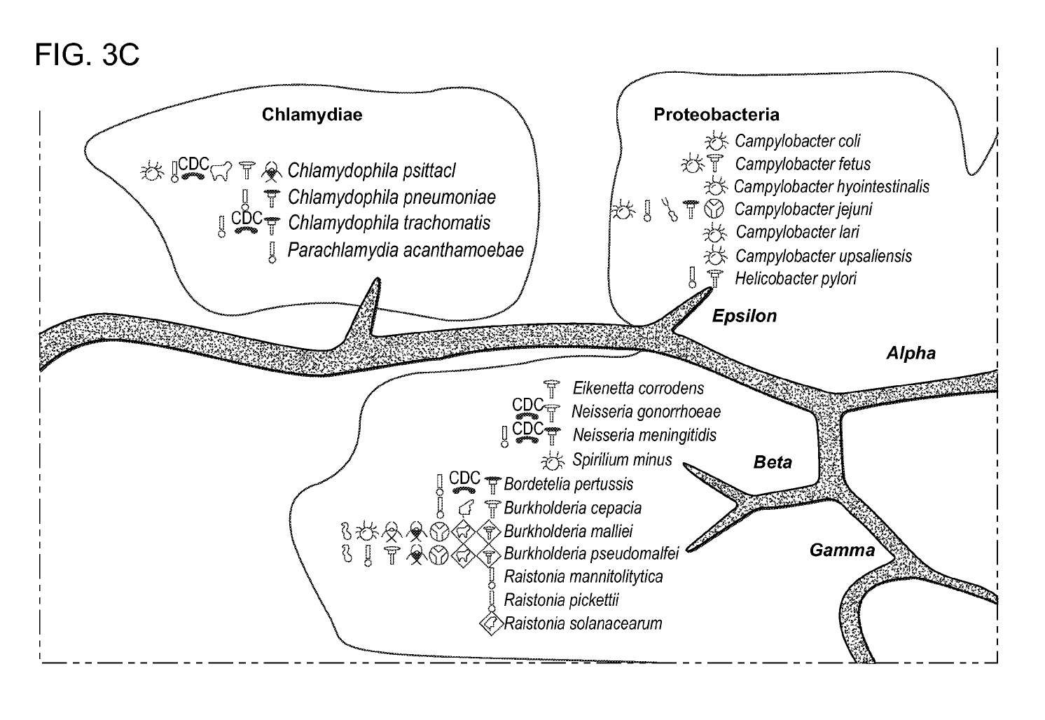

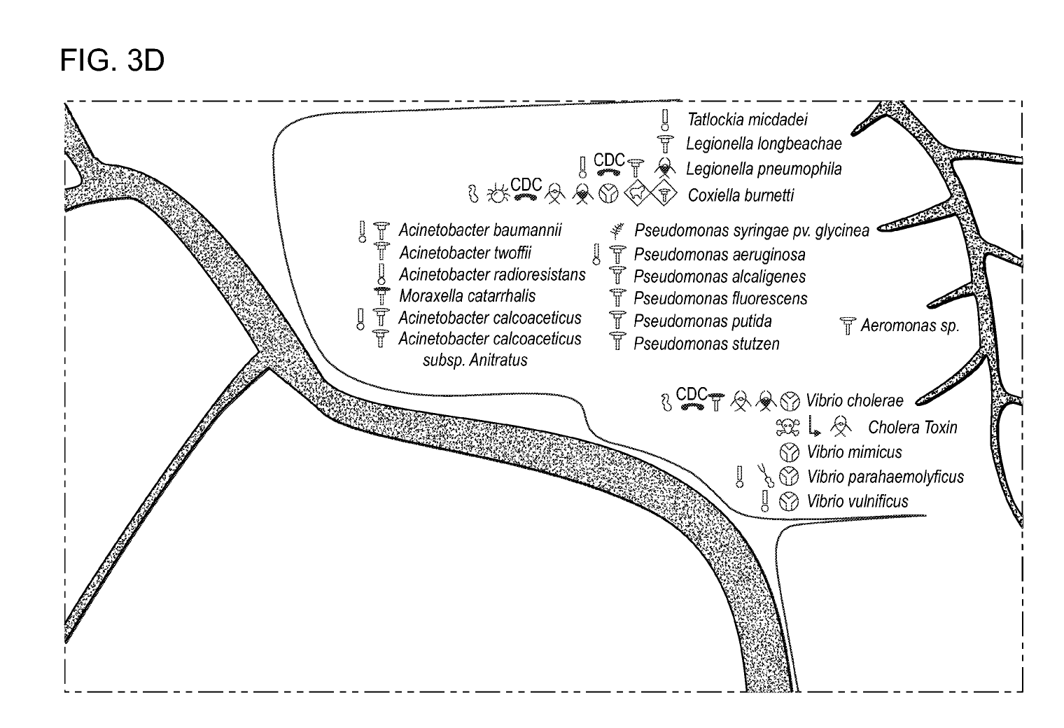

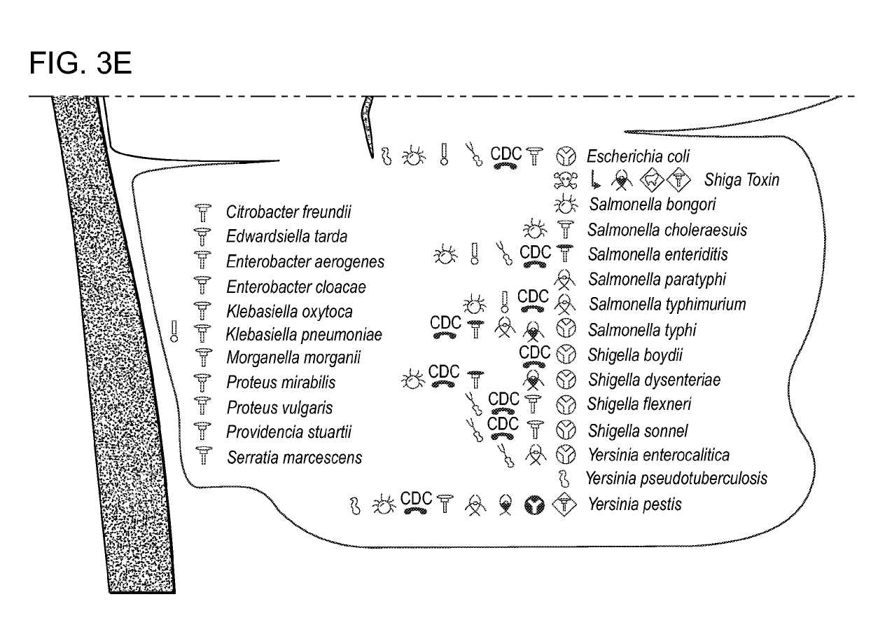

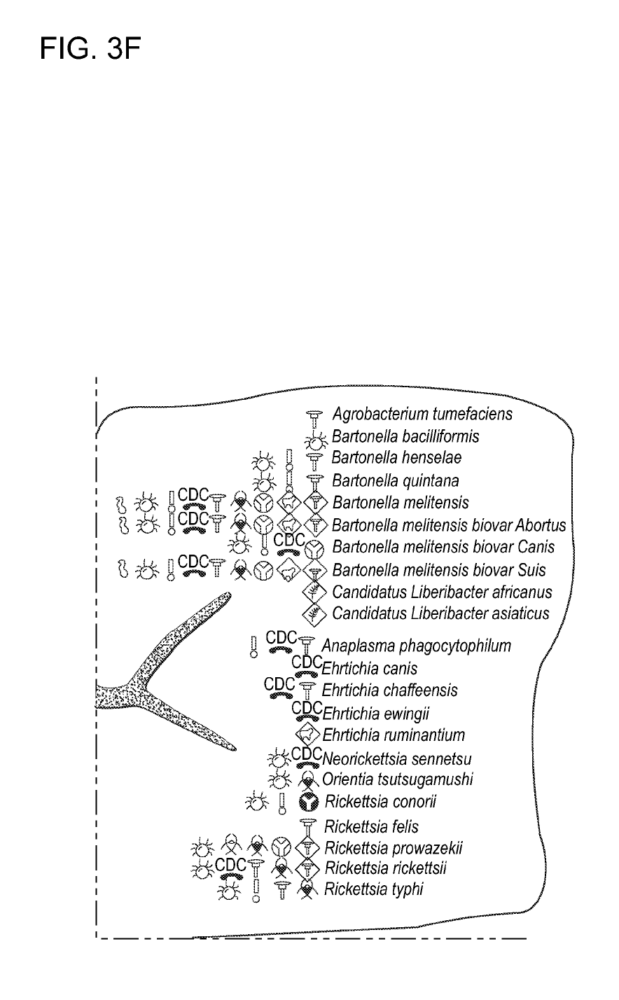

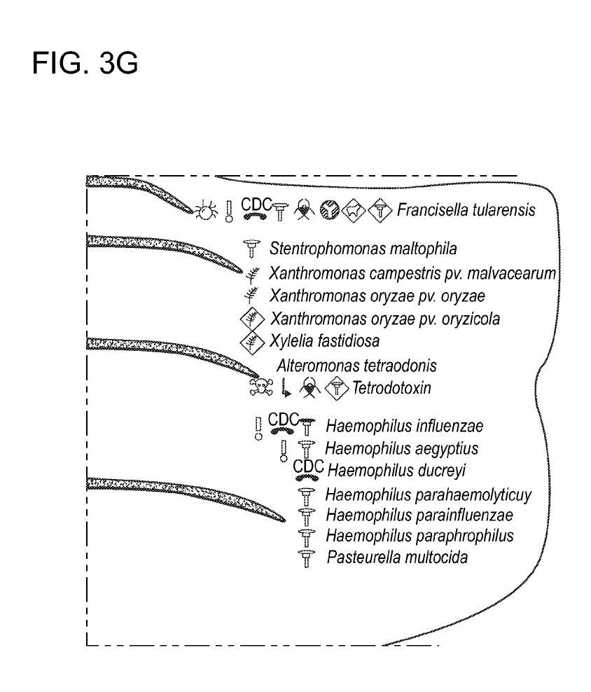

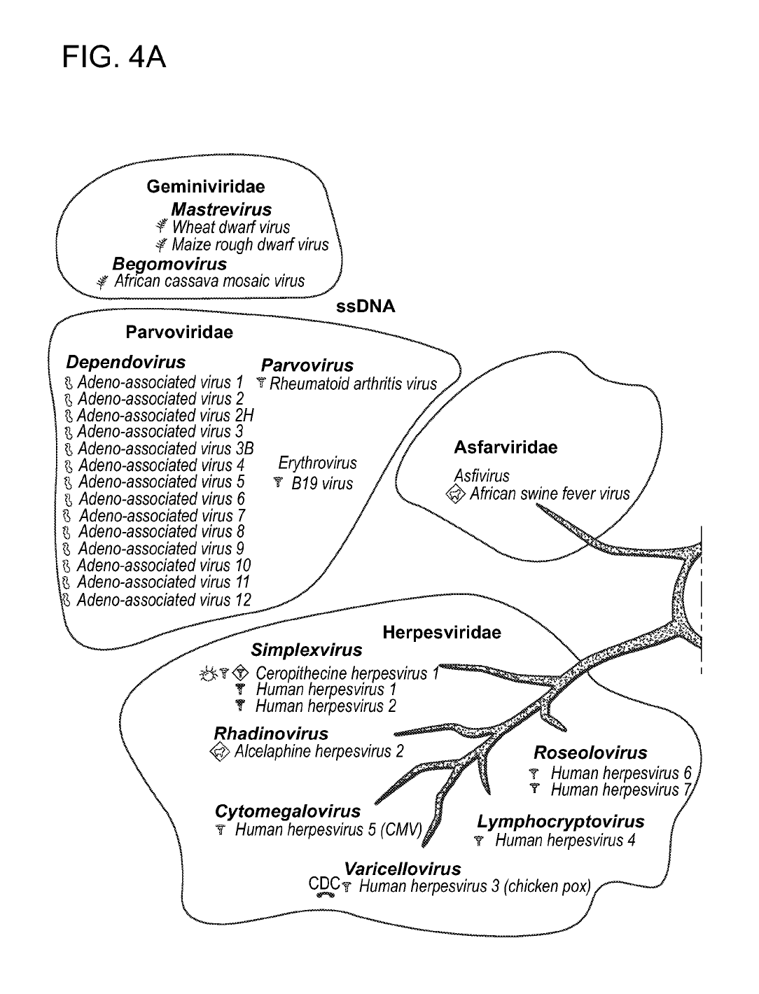

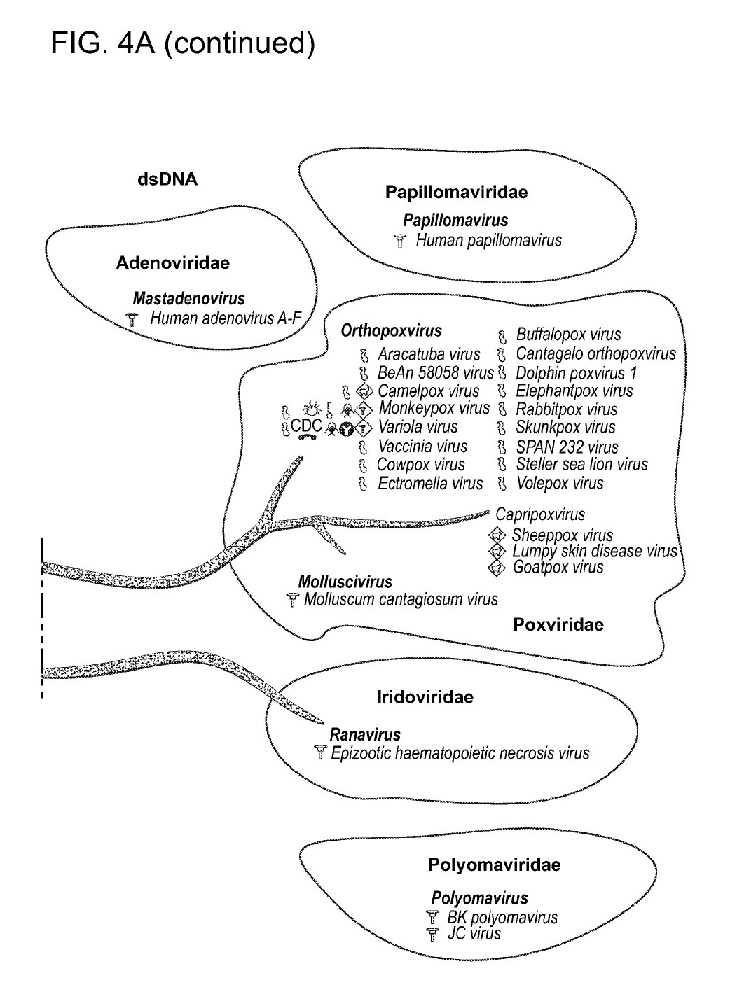

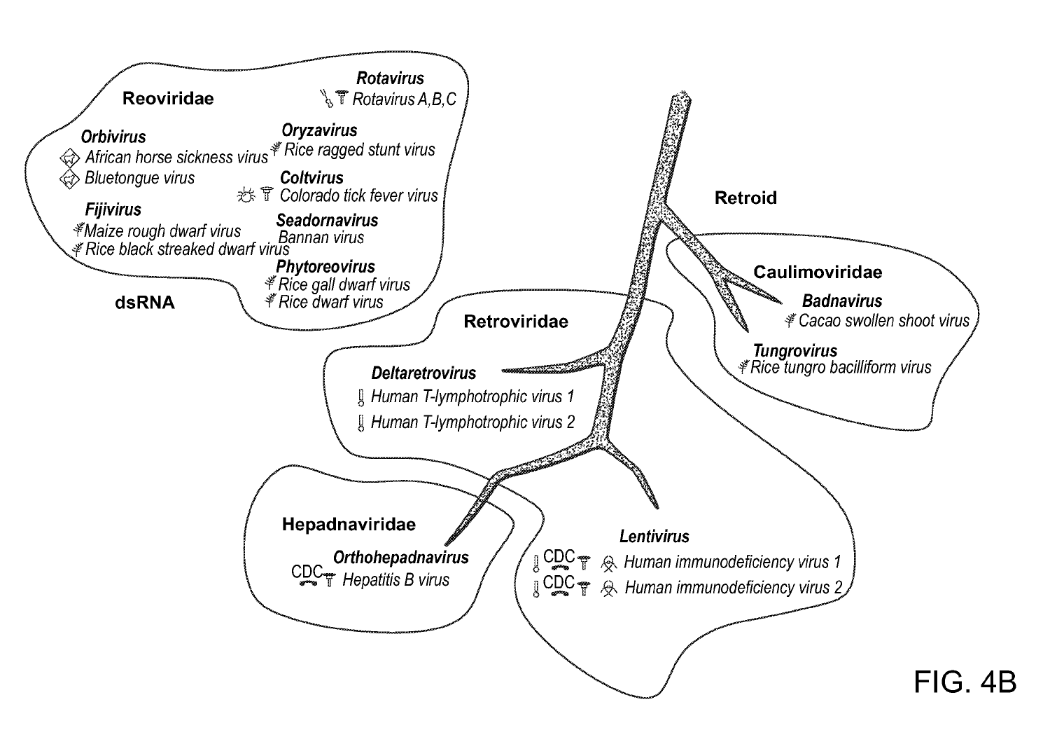

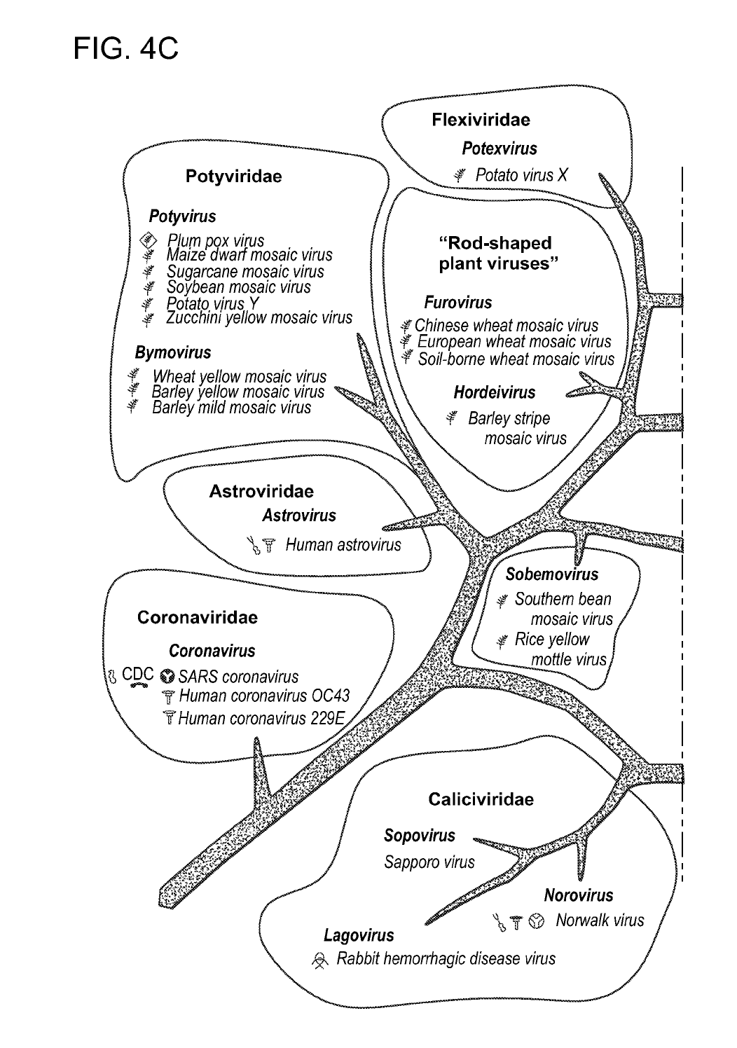

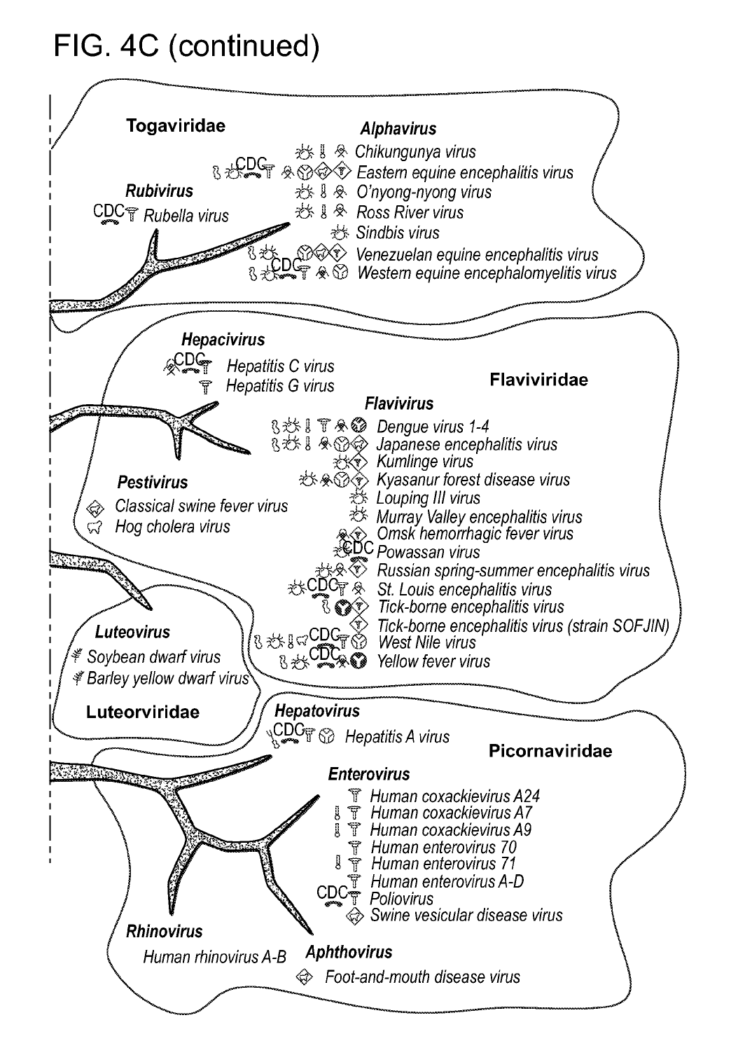

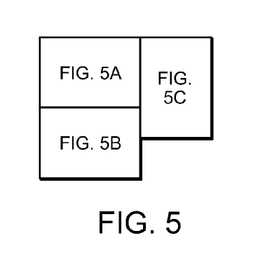

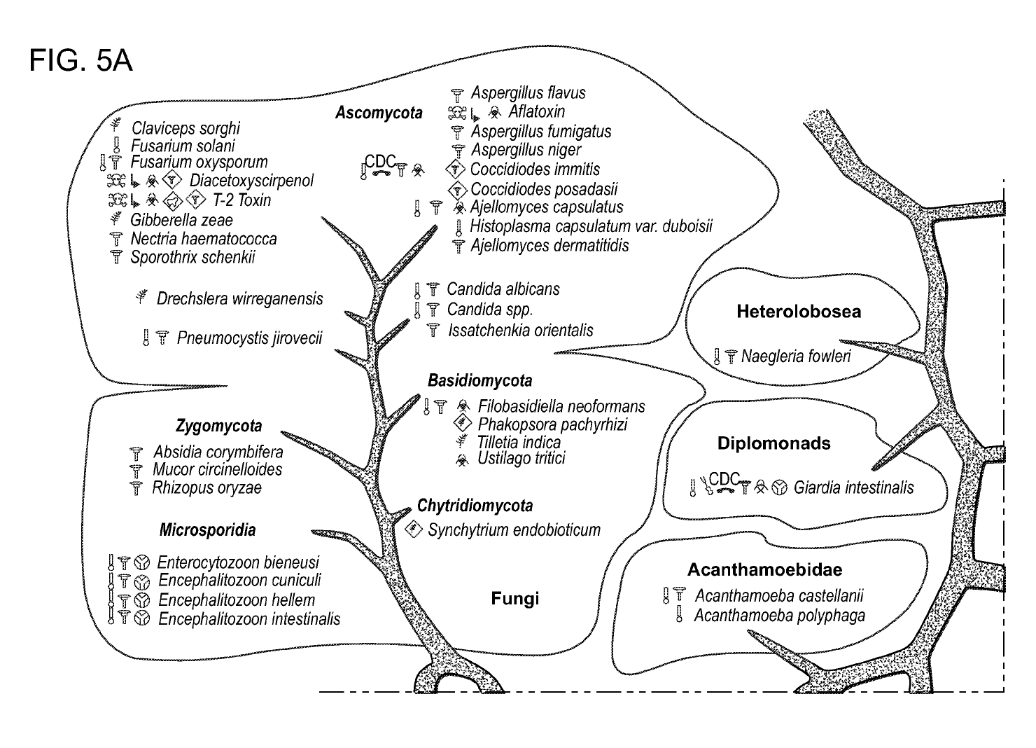

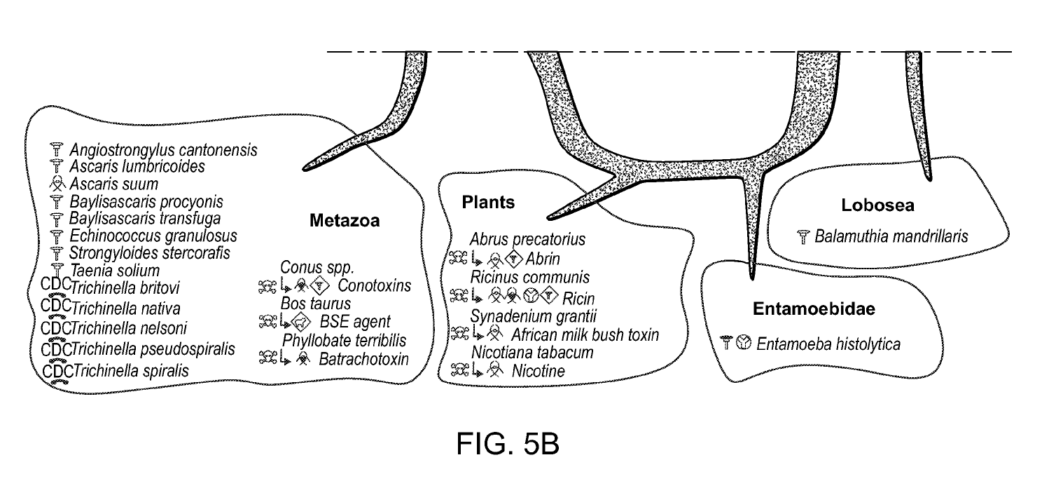

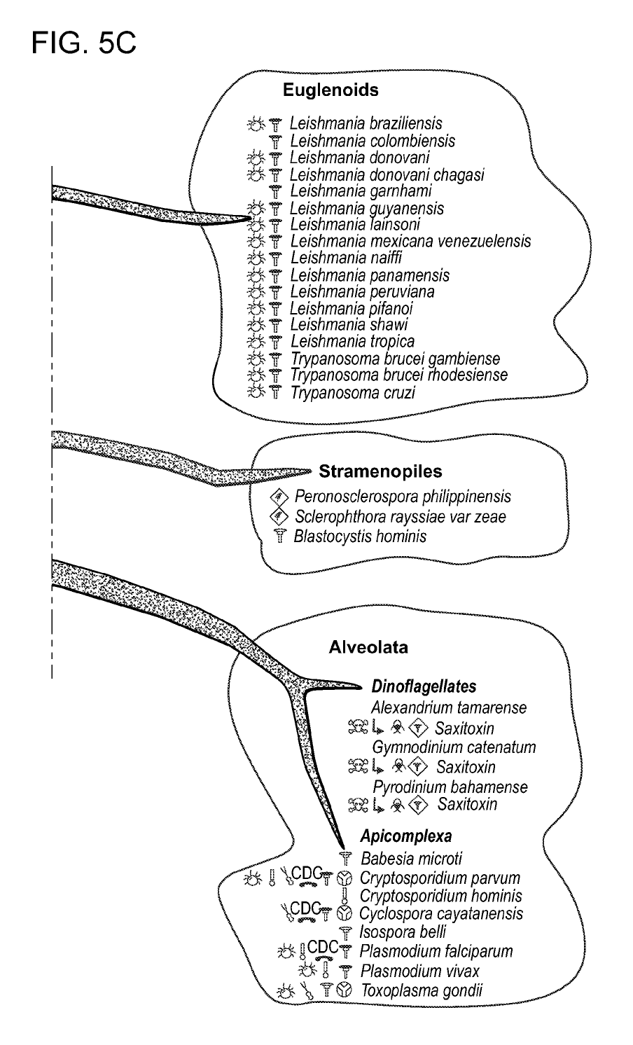

A large number of padlock probes (oligonucleotide probes that can circularize) can be used to specifically capture single nucleotide polymorphisms (SNPs) from genomic DNA, and the associated SNP identities and genotypes can be subsequently assessed by massively parallel DNA sequencing (FIG. 3). Without intending to be bound by theory, padlock probes likely provide the highest specificity among current genotyping methods because the circularization involves the combination of (i) cooperative annealing of two short sequences to a target in a uni-molecular fashion, (ii) allele-specific single-base extension, (iii)allele-specific ligation. In contrast, both Affymetrix's GENECHIP® and Illumina's INFINIUM™ assays involve a hybridization step that has an inherent limitation in distinguishing very similar sequences. Without intending to be bound by theory, padlock probes likely represent the best opportunity to further increase the number of SNPs determined in one assay from approximately 500,000 to approximately 10 million. Furthermore, combining padlock probes with DNA sequencing creates a distinct feature not possible with any of the current array-based methods: multiplexing on a large number of samples (Syvanen (2005) Nat. Genet. 37:S5-10). To achieve two dimensional (2D) genotyping, padlock probes circularized on different samples will be tagged with unique sample barcodes and pooled for DNA sequencing. The genotype at a given SNP locus of a certain sample will then be decoded by the combinations of three barcodes, allele barcode, locus barcode and sample barcode, all obtained in a single sequencing run. This provides an enormous advantage over existing technologies in that a single technology platform can be used for projects with a wide spectrum of SNP number and sample size combinations.

### EXAMPLE II

### Pathogen Polymorphism Database

A pathogen polymorphism database has been designed that compiles genetic signatures of 20 infectious disease pathogens and biosecurity threats. The database was compiled based on genomic regions that code for resistance, virulence, toxins and surface proteins. Regions of interest were shown to be unique to target pathogens in clinical and environmental mixtures.

The pathogen polymorphism database includes the pathogens set forth in Table 1.

TABLE 1

| | | CDC Bio-threat |
|---|---|---|
| Target | ID/EID/HAI | Category |
| Y. pestis | EID/ID | C |
| Yellow fever | ID | B |
| Brucella | ID | A |
| Avian pathogenic E. coli | EID | B |
| Quinolone resistant E. coli* | EID | B |
| Rickettsiae | EID/ID | A |
| Group B Streptococci | ID | N/A |
| Burkholderia mallie | ID | B |
| Bordetalla parapertusis | ID | N/A |
| Avian flu | ID | A |
| Dengue virus | EID | N/A |
| Drug resistant P. falciparum* | EID | N/A |
| M. tuberculosis* | EID/ID | C |
| V. cholera | ID | B |
| HIV-1* | ID | N/A |
| B. anthracis* | ID/EID | A |
| E. faecium | ID | N/A |
| F. tularensis | ID/EID | A |
| B. pertussis | ID | N/A |
| MRSA*# | HAI | N/A |

ID, infectious disease; EID, emerging infectious disease; HAI, hospital acquiredinfection;
*Wild-type and drug resistant variants;
#methicillin resistant S. aureus.

The methods an compositions herein are capable of identifying a pathogen's strain, genes encoding for antibiotic resistance and other virulence factors with superior dynamic range and precision. The methods and compositions described herein enable on of skill in the art the ability to identify pathogens in clinical and environmental samples without the need for culturing. Further, next generation sequencing-based technology will allow for the detection of drug resistant and more virulent strains, with a turn around time of 4-6 hours.

Databases can be designed to include mixtures of bacteria, viruses, fungi and parasites. Sample processing time will be reduced from four days to less than four hours by: 1) reducing hybridization time by increasing probe molecule concentration; 2) using sequencing primers as probe backbones instead of using PCR primer backbones; and 3) reducing sequencing time via multiplexing and barcoding. Multiplex processing of 200 samples in four to six hours will be performed. The database will be expanded to 100 pathogens or more. Blind identification of at least 20 infectious diseases will be performed from mixed samples.

Table 2 lists probes for detecting pandemic V. cholerae O1 and V. parahaemolyticus, as well as potentially pandemic V. cholerae O139.

TABLE 2

| | Predicted Protein | | Nucleotide |
|---|---|---|---|
| Gene Target | Function | Indicates | Position |
| rfbN | Biosynthesis of | Serogroup level V. | 40 |
| | O1 Antigen | Cholera | |
| wbfR | Biosynthesis of | Serogroup level V. | 9 |
| | O139 Antigen | Cholera n | |
| ctxA | Cholera toxin A | CTX-toxigenic | 85 |
| | subunit | isolate | |
| ctxB | Cholera toxin B | CTX-toxigenic | 37 |
| | subunit | isolate | |
| tl, tdh, VP1696, | Identification of | Serogroup level | |
| VPA1339, | characteristic toxin, | pandemic V. | |
| VPA1346, orf8, | type III secretion | parahaemolyticus | |
| O3:K6 spe, | system and | | |
| toxRS/new, | serotype-specific | | |
| HU-a ORF | markers for | | |
| | pandemic V. | | |
| | parahaemolyticus | | |
| | O3:K6 | | |

Table 3 lists probes for detecting vancomycin resistant Enterococcus faecium (*streptomycin (STR), ampicillin (AMP), kanamycin (KAN), tetracycline (TET), ciprofloxacin (CIP)).

TABLE 3

| | Predicted Protein | | Nucleotide |
|---|---|---|---|
| Gene Target | Function | Indicates | Position |
| vanA | VanA ligase | Selects for Vanc. A | 18 |
| | | resistant E. Faecum | |
| Van B consensus | VanB protein | Van B resistant E. | 9 |
| | | Faecum | |
| VanC-1 | Van C ligase | V resistance in E. | 20 |
| | | Flavescens | |
| vanSB2 | VanB protein | Vancomycin resistant | 20 |
| | | E. Faecium | |
| vanXB2 | VanB protein | Vancomycin resistant | 70 |
| | | E. Faecium | |
| VanYB2 | VanB protein | Vancomycin resistant | 83 |
| | | E. Faecium | |
| vanRB2 | VanB protein | Vancomycin resistant | 21 |
| | | E. Faecium | |

Table 4 lists probes for detecting B. anthracis based on genes encoding virulence factors.

TABLE 4

| Gene Target | Predicted protein Function | Indicates |
|---|---|---|
| capA | Capsule synthesis and | Presence of B. |
| | degradation | Anthracis |
| capB | Capsule synthesis and | Presence of B. |
| | degradation | Anthracis |
| lef | toxin synthesis | Presence of B. |
| | | Anthracis |

Table 5 lists probes for detecting multidrug resistant M. tuberculosis.

TABLE 5

| | | | Nucleotide |
|---|---|---|---|
| Gene Target | Mutation | Indicates | Position |
| Rv2043c | A/G | Pyrizinamide | 11 |
| | | Resistance | |
| Rrs | C/T, A/C, | Streptomycin | 491, 913, |
| | A/C | Resistance | 506 |
| Rv0005 | AAC/GAC | Fluoroqunolone | 1612 |
| | | Resistance | |
| Rv3795 | ATG/CTG | Ethambutol | 916 |
| | | Resistance | |
| Rv1908c | GGC/GAC | INH resistance | 836 |

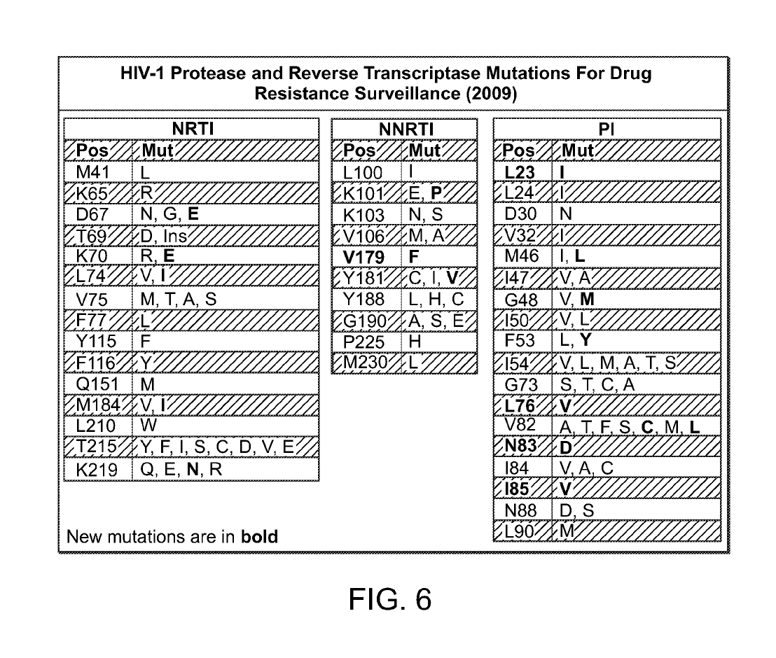

FIG. 6 depicts protease and reverse transcriptase mutations for drug resistance surveillance that existed in 2009 (new mutations are depicted in bold).

Table 6 depicts probes for detecting other targets.

TABLE 6

| Gene Target | Predicted protein Function | Selection Criteria |
|---|---|---|
| mecA | Penicillin binding protein | MRSA identification |

Table 7 depicts a comparison of methods known in the art to the methods described herein.

TABLE 7

| | | | | | Price | Blind ID | | |
|---|---|---|---|---|---|---|---|---|
| | | # of | Resolution | | (USD) per | in Complex | | MP |
| Company | Platform | Diseases | in Mixtures | Time | Sample | Mixture | Resistance | LX |
| Opgen | Optical | 40? | Species | 3 weeks | 2-3,000 | NO | NO | 0 |
| | mapping | | | | | | | |
| Cepheid | RT-PCR | 4 | Species | 90 minutes | 80 | NO | NO | 4 |
| | | | | (after | | | | |
| | | | | sample | | | | |
| | | | | culturing) | | | | |
| Focus DX | Culture/ | 50 | Species | 3-30 days | 298-500 | NO | NO | n/a |
| | RT-PCR/ | | | | | | | |
| | Antibody | | | | | | | |
| Affymetrix | 16s RNA | 0 | Species/ | 3-4 days | 200 | NO | NO | 0 |
| (Phylo- | sequence | | genus/ | | | | | |
| chip) | microarray | | taxa | | | | | |
| Methods | Nucleic | 20 | Subspecies/ | 3-4 days | 5 | YES | YES | 300 |
| and | acid | | Strain/ | (4-6 | | | | |
| Compositions | sequence | | serotype | hours in | | | | |
| Presented | (e.g., | | | future) | | | | |
| Herein | DNA, RNA) | | | | | | | |

### REFERENCES

Okou et al. (2007) Nat. Meth. 4:907

Albert et al. (2007) Nat. Meth. 4:903

Nilsson et al. (1994) Science 265:2085

Kurt et al. (2009) J. Clin. Microbiol. 47:577

What is claimed is:

1. A method for determining a phenotype of an organism in a sample comprising the steps of:
obtaining a sample;
contacting the sample with a molecular inversion probe (MIP), wherein the MIP includes two regions of homology to a target nucleic acid sequence of interest in the organism and two probe amplification regions, wherein the two regions of homology are selected using a MIP database specific for the phenotype;
hybridizing the MIP to the nucleic acid sequence of interest;
converting the target nucleic acid sequence of interest to circular DNA;
amplifying the circular DNA;
releasing the MIP from the amplified DNA;
sequencing the amplified DNA; and
determining whether a DNA sequence corresponding to the phenotype is present.

2. The method of claim 1, wherein the organism is selected from the group consisting of a bacterium, a virus, a fungus and a protist.

3. The method of claim 2, wherein the bacterium is selected from the group consisting of Y. pestis, Brucella, Avian pathogenic E. coli, Quinolone resistant E. coli, Rickettsiae, Group B Streptococci, Burkholderia mallie, Bordetalla parapertusis, drug resistant P. falciparum, M. tuberculosis, V. cholera, B. anthracis, E. faecium, F. tularensis, B. pertussis and methicillin resistant S. aureus.

4. The method of claim 2, wherein the virus is selected from the group consisting of HIV-1, avian influenza and dengue virus.

5. The method of claim 1, wherein the amplification step is performed by rolling circle amplification (RCA).

6. The method of claim 1, wherein the sequencing step is performed by multiplex sequencing.

7. The method of claim 1, wherein the MIP database is a single nucleotide polymorphism (SNP) database.

8. The method of claim 1, wherein the MIP database is an antibiotic resistance gene database.

9. The method of claim 1, wherein the MIP database is a virulence gene database.

10. The method of claim 1, wherein the phenotype is antibiotic resistance.

11. The method of claim 1, wherein the phenotype is virulence.
